(19) United States
(12) Patent Application Publication
(10) Pub. No.: US 2015/0065822 A1
(43) Pub. Date: Mar. 5, 2015

(54) SYSTEMS AND METHODS FOR ADDICTION PREVENTION AND TREATMENT

(71) Applicant: NATHAN A. BLENKUSH, Attleboro, MA (US)

(72) Inventor: NATHAN A. BLENKUSH, Attleboro, MA (US)

(21) Appl. No.: 14/474,579

(22) Filed: Sep. 2, 2014

Related U.S. Application Data
(60) Provisional application No. 61/873,979, filed on Sep. 5, 2013.

(51) Int. Cl. A61B 5/00; A61M 5/172; A61B 5/145

(52) U.S. Cl. CPC A61B 5/4845; A61B 5/14503; A61B 5/4839; A61B 5/686; A61B 5/746; A61B 5/14546; A61M 5/1723; A61B 5/486; A61B 5/7282; A61B 5/6898; A61M 2230/005; A61M 2205/50
USPC 600/309; 600/300

(57) ABSTRACT

The invention pertains to systems and methods for preventing or reducing the probability of human addiction to various substances and/or treating those currently or previously addicted to substances. The invention includes a sensing mechanism to detect a presence of the addictive substance or a condition resulting from the presence of the addictive substance, a signaling mechanism to generate a signal as a result of the sensing mechanism detecting the presence of the addictive substance or a condition resulting from the presence of the addictive substance, and a receiving mechanism to provide at least one response as a result of receiving as input the signal generated by the signaling mechanism

### CROSS-REFERENCE TO RELATED APPLICATIONS

This application claims priority under 35 U.S.C. §119(e) to U.S. Provisional Patent Application Ser. No. 61/873,979, filed Sep. 5, 2013, entitled “Systems and Methods for Addiction Prevention and Treatment”, which is herein incorporated by reference.

### FIELD OF THE INVENTION

The invention pertains to systems and methods for precluding or reducing the probability of addiction by humans to various substances and/or treating humans currently addicted or having a history of substance addiction.

### BACKGROUND INFORMATION

Addictive substances can cause extraordinary human suffering. For example, use of and/or addiction to drugs, tobacco, or alcohol can lead to premature death of a person through overdose, disease, or accident. Addiction to such substances can separate children and parents, and destroy marriages, friendships, and productive human behavior. In 2004, The National Institutes of Health estimated that drug, alcohol, and tobacco addiction cost the United States around $500 billion per year. Despite human knowledge regarding the ill effects of addiction or exposure to substances such as nicotine, alcohol, opiates, stimulants, and other substances, addiction remains an extraordinary problem in many societies around the world.

Addiction can be described using several behavioral principles. For instance, drug effects have been viewed as unconditioned stimuli. When a human is exposed to a drug, stimuli associated with the use of the drug (smells, tastes, sight of drug paraphernalia, etc) can become conditioned stimuli through classical conditioning. Exposure to the drug and associated stimuli that accompany the drug can become conditioned through classical conditioning and as a result can acquire functions (e.g., the power to reinforce, decelerate, and otherwise stimulate various behaviors) similar to those that the drug possesses. Thereafter, both those conditioned stimuli, as well as the drug itself, can function as reinforcers, decelerators, or stimuli for various voluntary (operant) and involuntary (reflexive) behaviors. Typically, initial use of many drugs produces what drug users subjectively label, a “high” or euphoria. Continued use of many drugs leads to behavioral and neurobiological alterations which are well known in the art, such as escalation in drug intake, impulsive or compulsive drug use, markedly increased cravings, and withdrawal symptoms. Thus, initial drug administering behaviors may be positively reinforced increasing the probability of similar behavior occurring in the future. Over time with continued use and even with attempts to cease using the drug, withdrawal symptoms arise. Drug administering behaviors then may be negatively reinforced by decreasing, terminating, or precluding the physiological state (that is subjectively unpleasant) caused by the absence of the drug.

Numerous procedures have been developed to prevent and treat addiction to various substances by humans. Regarding prevention, educational procedures aimed at people at risk, taxing of legal drugs, and punitive procedures (directed at users, sellers, traffickers, producers) have been implemented to reduce the probability of addiction. In addition, attempts to address the issue of supply have been made through the use of military forces and improved border security.

Known treatments to address addiction include medications and psychosocial treatments. Medications may be utilized to suppress withdrawal symptoms during detoxification. Pharmacological treatments such as methadone, bupropion, varenicline, naltrexone, and acamprosate may be employed to address addictions to various substances. Psychosocial treatments such as family therapy, cognitive-behavioral therapy, and residential treatment with various theoretical approaches have been developed. Often times, these treatments are voluntary in nature and addicts are willing to undergo such treatments only after the addiction has caused significant damage or tragedy in their lives.

The early use of counter-conditioning to treat existing addictions is also known in the art. Various publications have described the short- and long-term success of the procedure. Disulfiram has been long known and used to treat addiction to alcohol. However, because patients can simply choose not to ingest the drug, efficacy has been inconsistent.

There is a need and desire in the art to prevent addiction from occurring. It is contemplated that by preventing the initial exposure to or use of a given addictive substance, the cycle of positive and negative reinforcement will not ensue. The disease processes caused by a particular substance are arrested and the cascade of destructive life events (e.g. relationship destruction, job loss, economic ruin, acts of crime such as theft and prostitution) may be prevented. For those persons who are addicted, it is important to maintain an immediate and sufficiently aversive consequence that outweighs the reinforcing value of the addictive substance. Alternatively, eliminating the positively reinforcing effects of a drug upon administration may also aid in long-term recovery.

It is further contemplated that reducing the number of addicts decreases the financial reinforcers for addictive substance production, sales, and trafficking. The availability of such substances then decreases as does the probability one may encounter an addictive substance.

### SUMMARY OF THE INVENTION

In one aspect, the invention provides a system for detection and treatment of use of an addictive substance by a human. The system includes a sensing mechanism to detect a presence of the addictive substance or a condition resulting from the presence of the addictive substance in a body of the human, a signaling mechanism to generate a signal as a result of the sensing mechanism detecting the presence of the addictive substance or a condition resulting from the presence of the addictive substance, and a receiving mechanism to provide at least one response as a result of receiving as input the signal generated by the signaling mechanism. The at least one response is selected from the group consisting of identifying the presence of the addictive substance or condition, releasing a drug effective to induce an aversive physical condition, releasing a drug to counteract effects of the addictive substance, and depolarizing neurons through chemical or electrical action.

The sensing mechanism can include one or more sensors selected from the group consisting of implantable sensors and transdermal sensors.

The receiving mechanism can include a drug delivery device. The drug delivery device may be selected from the group consisting of an implantable medical device, a transdermal device and a injector device.

The sensing mechanism can detect the presence of the addictive substance or condition when the addictive substance or condition meets or exceeds a pre-selected threshold value.

The receiving mechanism can quantify the addictive substance or condition in the body. The receiving mechanism can be a monitoring device positioned external to the body of the human that produces an alarm which is indicative of the presence of the addictive substance or condition.

The condition resulting from the presence of the addictive substance may be selected from the group consisting of atoms, ions, molecules, or molecular assembly of the addictive substance; physiological response to the addictive substance and metabolite of the addictive substance.

The system of the invention can further include behavioral intervention.

The sensing, signaling and receiving mechanisms can serve as an addiction prevention device.

In another aspect, the invention provides a method for detecting and treating use of an addictive substance by a human. The method includes identifying a human that uses or has a likelihood to use the addictive substance, administering to a body of the human a sensing mechanism that detects a presence or a pre-selected threshold level of the addictive substance or condition resulting from the presence of the addictive substance in the body, connecting the sensing mechanism to a signaling mechanism, producing and sending at least one signal when the sensing mechanism detects the presence or pre-selected threshold level of the addictive substance or condition resulting from the presence of the addictive substance in the body, and receiving the signal(s) and providing at least one response. The response(s) selected from the group consisting of generating an alert or indication of the presence of the addictive substance or the condition in the human body, releasing a drug effective to induce an aversive physical condition, releasing a drug to counteract effects of the addictive substance, and depolarizing neurons through chemical or electrical action.

The releasing of the drug may consist of employing an implanted drug delivery device in combination with a computer such that one or more doses of the drug is released from the device in response to the computer sending a command The computer can include a portable device selected from the group consisting of a smartphone and tablet.

The method of the invention can further include providing a diagnosis and treatment relating to use of the addictive substance by the human

The one or more doses can be selected from the group consisting of a one-time dose, a continuous dose, an intermittent dose and a dose on an as-needed basis.

### DETAILED DESCRIPTION OF PREFERRED EMBODIMENTS

The invention relates systems and methods for preventing or reducing the probability of human addiction to various substances and/or treating those currently or previously addicted to substances. A wide variety of addictive substances are known in the art and include, but are not limited to, recreational and prescription drugs, alcohol, and tobacco. Non-limiting examples include opiates such as morphine and codeine, opioids such as heroin and oxycodone, cocaine, amphetamines and methamphetamines In certain embodiments of the invention, systems and methods are provided for detecting and monitoring the intake of addictive substances, notifying a third party, such as medical personnel, of the intake of addictive substances, and initiating a response to counteract the addictive substances or to reduce the likelihood of continued intake of the addictive substances.

The invention includes sensing means, signaling means, and receiving means which are connected. These means may be wired to each other or they may be connected wirelessly, according to technology known in the art.

The sensing means detects the presence of an addictive substance in the body of a human, e.g., patient, and can include a wide variety of sensing mechanisms and devices that are known in the art. Non-limiting examples include one or more implantable sensors or transdermal sensors which are effective to detect the presence of an addictive substance in the body. The implantable sensor(s) consist of medical implant devices which are known in the art. Implanted sensor(s) are constructed and selected based on particular necessary or desirable characteristics. For example, the implanted sensor(s), e.g., electrochemical-based biosensors, may be composed of a material that is adaptive to biological/physiological environments and does not result in adverse effects to the human body, e.g., materials that are biocompatible. In addition, the implanted sensor(s) may also be biodegradable.

In certain embodiments, the sensor(s) detect the mere presence or absence of an addictive substance and in certain other embodiments, the sensor(s) may detect a level, concentration or degree of the addictive substance in the human body. For example, the sensor(s) may be programmed to detect the presence of an addictive substance that meets or exceeds a pre-selected threshold value.

The transdermal sensor(s) may be applied to the exterior surface, e.g., skin, of the human body. In these embodiments, the sensor(s) is constructed or composed of a material that is adaptive to the skin surface of the human body such that there is no resulting irritation or injury.

Sensors known in the art are capable of detecting, for example, heroin, noscapine, and morphine at micromolar concentrations using graphene nanosheets. There are also known sensors which employ a biological nanopore embedded in a microchip that can detect cocaine. Further, known sensors consisting of a protein array chip are capable of detecting cocaine, ecstasy, heroin, and amphetamine using imaging surface plasmon resonance. Furthermore, there are known transdermal alcohol content monitoring sensors or devices that detect the presence of alcohol. There has even been described in the art the use of frog melanophores for detection of opioids. Any of these sensors can be used in accordance with the invention to determine the presence or absence or level/degree/concentration of an addictive substance in the body of a human, e.g., patient.

In addition to detecting and monitoring the presence/absence and level/degree/concentration of addictive substances, sensors may be used to detect and monitor responses caused by an addictive substance in the body of the patient. Typically, addictive substances may produce one or more of the following responses or conditions that are detectable by the sensors:
Atoms, ions, molecules, or molecular assembly of an addictive substance;
Physiological response to an addictive substance; and/or
Metabolite of an addictive substance.

The sensor(s) may detect the mere presence of the above conditions or the sensor(s) may detect a level, concentration or degree of the above conditions. The level, concentration or degree may be compared to a control. The control may be the level, concentration or degree associated with the absence of an addictive substance or the control may be a pre-selected threshold value or condition.

The signaling means generates, transduces and transmits a signal as a result of the sensing means detecting the presence of an addictive substance or condition in a human body, or detecting an addictive substance or condition equal to or above a pre-selected or pre-determined threshold value. The signaling means can include various signaling or transducing mechanisms and devices that are known in the art. The signal is then transmitted to a receiving means.

The receiving means receives as input the signal produced by the signaling means and generates a response. The receiving means can include various receiving mechanisms or devices that are known in the art. The response generated can vary and depends on the receiving device employed. In certain embodiments, the receiving means can be effective to merely identify or notify that an addictive substance or condition is detected in the body of the patient. For example, the receiving means can display an output showing the determination of the presence or absence of an addictive substance or condition, or the determination that a pre-selected threshold value is met or exceeded, or the receiving means can display the type of addictive substance or condition that is detected, or the receiving means can display and specify the level/concentration/degree of the addictive substance or condition detected, or the receiving means can generate/sound an alarm indicative of the mere presence of the addictive substance or condition, or its presence at or above a pre-selected threshold value.

Alternatively or in addition to the display and/or alarm functions, the response provided by the receiving means can include further action to address the addictive substance or condition detected in the body of the patient.

In certain embodiments, the signal from the signaling means is sent to and received by a drug delivery device which is capable of providing one or more of the following responses:
Release of a chemical or drug that induces nausea and/or some type of unpleasant state into the body;
Release of a chemical or drug that immediately counteracts the effect(s) of the addictive substance; and
Depolarization of neurons through chemical or electrical action.

Various chemicals and drugs that induce nausea or an unpleasant state in the body are well known in the art and include, for example, e.g., apomorphine. Suitable chemicals and drugs for use in the invention include those that are safe, e.g., do not cause any known risk to the health and safety of the patient, but provide an unpleasant feeling or condition in the patient such that the patient is discouraged from repeating (or less likely to repeat) an intake of the addictive substance in order to preclude the unpleasant feeling or condition.

Further, various chemicals and drugs that counteract the effect of an addictive substance are well known in the art and include, for example, e.g., naloxone. Suitable chemicals and drugs for use in the invention include those that are safe, e.g., do not cause any known risk to the health and safety of the patient, but inhibit or preclude the pleasant feeling or condition that is typically associated with addictive substance or sought after by the patient as a result of taking the addictive substance. Without intending to be bound by any particular theory, it is believed that a patient will be less likely to take an addictive substance if its effect is inhibited or precluded such that the patient does not experience the pleasant feeling or condition that he/she associated with taking the addictive substance.

The chemicals and drugs can be administered to the patient using various drug delivery devices that are known in the art. For example, the chemicals and drugs can be administered by the patient or a third party, such as a medical professional, by injection into the body of the patient using, for example, a syringe, or the doses can be injected using an intravenous device. Further, in certain embodiments, the chemicals or drugs can be provided by employing implantable medical devices and transdermal medical devices. Implantable medical delivery devices are routinely used in many medical specialties and manufacturers of these devices incorporate various chemicals or drugs into the medical implants for release into the human body. For example, implantable micro-chip-based drug delivery devices are known in the art. The release of the drug can be targeted to a particular location and can be controlled, such as according to a particular dosing schedule. The dosing schedule can include a one-time dose, a continuous dose, an intermittent dose or a dose on an as-needed basis. In certain embodiments, the release of chemicals or drugs in the body can be controlled by employing microchip-based devices that are programmed to release intermittent and/or discrete doses of a drug, such as once daily for a period of days.

A non-limiting example of an implantable drug delivery system includes a medical implant device connected to (e.g., wired or wireless) with a computer. The computer can be a PC-based computer or a mobile computer, such as hand-held device, e.g., smartphone. The implant device may be a microchip assembly including control and communication electronics. The implant device may also have the ability to contain one or more doses of a drug. The drug dose may be released upon receipt of a command from the computer or the computer can be programmed such as to provide a pre-determined dose at a pre-specified time in the future.

In certain embodiments, a computer-based programmer operating in the Medical Implant Communications Service band may establish a bidirectional wireless communication link with the medical implant device to program the dosing schedule and receive implant status confirming proper operation.

Another suitable implantable drug delivery system for use in the invention can include a medical implant device connected to a patch, for example, located on top of the skin in the body area where the implant is placed. The patch carries out the power transmission, the data receiving and the data retransmission onto longer distances. The patch can communicate via Bluetooth connection to portable devices, such as smartphones, tablets or external control stations. A doctor, for example, can remotely receive the information through a dedicated interface on a smartphone or tablet. Otherwise, data can be stored in the memory of the phone or laptop and subsequently analyzed.

As indicated above, in lieu of the release of chemicals or drugs, a device could depolarize groups of neurons and cause an unpleasant bodily state such as nausea.

In alternative embodiments, an active or passive signal can be sent from the signaling means to a scanner or receiver mechanism which is located external to the human body, for example, in a home or rehabilitation environment. In certain embodiments, a first signal can be sent to a drug delivery device to activate the administration of the chemical or drug to the human (as described above), and a second signal, e.g., an alarm, can be sent to a scanner or receiver located in a home or facility to notify family, caregivers, and/or treating professionals that the presence of an addictive substance is detected.

In certain other embodiments, the creation of a signal or method of determining by scanning (such as through RFID technology) that a person was exposed to an addictive substance may be employed.

The above conditions and responses through principles of operant and/or classical conditioning are effective to provide one or more of the following outcomes:
Prevent or reduce the probability of addiction to the substance;
Signal caregivers that psychosocial or other interventions are indicated;
Discourage humans from experimenting with addictive substances;
Treat those currently addicted; and
Prevent or reduce the probability of relapse in those previously addicted.

In certain embodiments, a behavioral method of addiction prevention and treatment is employed which includes the internal implantation and/or external application of sensors, the internal implantation and/or external application of a drug delivery device or related device and, wireless and/or wired and/or chemical signaling

For those who are not addicted to a substance, the systems of the invention can serve as an addiction prevention device (APD) to reduce the probability of drug experimentation through rule-governed behavior. Rule-governed behavior is acquired through descriptions of, rather than through direct contact with, contingencies. For example, a patient is administered the sensing, signaling and receiving means of the invention. Sensor(s) may be implanted with the body of the patient as an APD. The APD-implanted patient is told that adding targeted addictive substances to his/her body will produce periods of nausea and/or sickness. Similarly, if the substance released from the device counteracts an addictive substance (such as the effect of naloxone on heroin), the patient may be less inclined to use the drug in the absence of euphoric effects.

For the addict and recovering addict, similar effects may be observed. Specifically, the prospect of coming into contact with the unpleasant bodily state may reduce the probability of relapse following treatment and may deter an addict from administering the drug again.

For the patient with an APD, the behaviors leading to the ingestion of the drug may be less likely to occur because of the introduction of substances inducing nausea and/or unpleasant bodily states. Operant punishment is the presentation of a stimulus following a response that reduces the probability of similar behavior occurring in the future. In addition, stimuli associated with the addictive substance (e.g. sight, smell, taste, etc) may become conditioned aversive stimuli through pairing with the unconditioned aversive stimuli (nausea inducing substance). As a result, the person may avoid such stimuli and avoid future contact with the addictive substance. Such avoidance responses may persist for substantial periods of time and in some cases, may persist indefinitely.

An APD that releases a substance that counteracts the effects of an addictive substance may reduce drug administrating behaviors through extinction. Operant extinction is the operation of withholding a reinforcing stimulus following a response. In general, operant extinction results in a momentary increase in response followed by a decrease in response over time.

In the case of someone who is exposed to an addictive substance for the first time and immediately treated with the APD, caregivers will also be able to implement behavioral programs to prevent subsequent exposure and solve family, personal, or issues related to skill deficits. Behavioral intervention involves using the principles of learning (e.g. reinforcement, punishment, stimulus control, schedule control, verbal behavior, extinction) to remediate or modify behavior for the good of mankind. For the recovering addict, the alert may also indicate such or other more intensive interventions.

In certain embodiments, the methods of the invention include identifying a human, e.g., patient, that has used or uses or has the likelihood to use an addictive substance and administering a sensing mechanism, e.g., one or more sensors, that is capable to detect the presence or a pre-selected threshold level of the addictive substance or a condition in the body of the human. The identification of the human can be based on medical records, current behavior or practices, past behavior or practices, individual or family history, and the like. The sensing mechanism may be implanted into the body of the human being and/or applied externally to the body, i.e., placed/adhered on the skin. The sensing mechanism may be connected to a signaling mechanism, e.g., transducer, such that when the sensing mechanism detects the mere presence of an addictive substance or condition in the human body or the presence of an addictive substance or condition in the human body that meets or exceeds a pre-selected threshold level, the signaling mechanism generates and sends at least one signal. The signal can be input to one or more receiving devices that provide a response to identify the detection and/or counteract the addictive substance and/or produce a negative physical condition, e.g., nausea. In certain embodiments, the receiving device includes a drug delivery device which releases a chemical or drug into the body to produce an effect, such as counteracting the addictive substance (e.g., inhibiting the expected pleasant feeling) or inducing nausea. The drug delivery device can be positioned inside the body, e.g., implanted, or outside the body, e.g., contacted with the skin In certain embodiments, the drug or chemical can be delivered or administered by human interaction by administering orally or by injection the drug into the human body of the patient. Further, the signaling device can include or be connected to a display mechanism or device which shows or quantifies the level or concentration or degree of addictive substance detected in the human body. The communications between the sensing, signaling, receiving and display mechanisms can be include wired or wireless connections.

Optionally, the signals generated and the responses produced can be monitored and recorded such that a determination or diagnosis as to the addictive state of the human being can be made. There are various instances wherein the addictive condition of a human being may be required. These instances can include the following: hiring a person for employment, allowing a person to have custody of a child, determining whether hospitalization is necessary, and the like. The number of signals and/or responses generated can be monitored by employing a counting and/or recording mechanism selected from those known in the art.

Further, data related to the number of signals and responses generated can be useful in determining whether continued treatment is needed or the particular level of treatment that is necessary.

Whereas particular embodiments of the invention have been described herein for purposes of illustration, it will be evident to those skilled in the art that numerous variations of the details may be made without departing from the invention as set forth in the appended claims.

In the claims:

1. A system for detection and treatment of use of an addictive substance by a human, the system comprising:
a sensing mechanism to detect a presence of the addictive substance or a condition resulting from the presence of the addictive substance in a body of the human;
a signaling mechanism to generate a signal as a result of the sensing mechanism detecting the presence of the addictive substance or a condition resulting from the presence of the addictive substance; and
a receiving mechanism to provide at least one response as a result of receiving as input the signal generated by the signaling mechanism, the at least one response selected from the group consisting of identifying the presence of the addictive substance or condition, releasing a drug effective to induce an aversive physical condition, releasing a drug to counteract effects of the addictive substance, and depolarizing neurons through chemical or electrical action.

2. The system of claim 1, wherein the sensing mechanism includes one or more sensors selected from the group consisting of implantable sensors and transdermal sensors.

3. The system of claim 1, wherein the receiving mechanism comprises a drug delivery device.

4. The system of claim 3, wherein the drug delivery device is selected from the group consisting of an implantable medical device, a transdermal device and a injector device.

5. The system of claim 1, wherein the sensing mechanism detects the presence of the addictive substance or condition when the addictive substance or condition meets or exceeds a pre-selected threshold value.

6. The system of claim 1, wherein the receiving mechanism quantifies the addictive substance or condition in the body.

7. The system of claim 1, wherein the receiving mechanism is a monitoring device positioned external to the body of the human that produces an alarm which is indicative of the presence of the addictive substance or condition.

8. The system of claim 1, wherein the condition resulting from the presence of the addictive substance is selected from the group consisting of atoms, ions, molecules, or molecular assembly of the addictive substance; physiological response to the addictive substance and metabolite of the addictive substance.

9. The system of claim 1, further comprising behavioral intervention.

10. The system of claim 1, wherein the sensing, signaling and receiving mechanisms serve as an addiction prevention device.

11. A method for detecting and treating use of an addictive substance by a human, the method comprising:
identifying a human that uses or has a likelihood to use the addictive substance;
administering to a body of the human a sensing mechanism that detects a presence or a pre-selected threshold level of the addictive substance or condition resulting from the presence of the addictive substance in the body;
connecting the sensing mechanism to a signaling mechanism;
producing and sending at least one signal when the sensing mechanism detects the presence or pre-selected threshold level of the addictive substance or condition resulting from the presence of the addictive substance in the body; and
receiving the at least one signal and in response, providing at least one response selected from generating an alert or indication of the presence of the addictive substance or the condition in the human body, releasing a drug effective to induce an aversive physical condition, releasing a drug to counteract effects of the addictive substance, and depolarizing neurons through chemical or electrical action.

12. The method of claim 11, wherein releasing the drug consists of employing an implanted drug delivery device in combination with a computer such that one or more doses of the drug is released from the device in response to the computer sending a command.

13. The method of claim 12, wherein the computer comprises a portable device selected from the group consisting of a smartphone and tablet.

14. The method of claim 11, further comprising providing a diagnosis and treatment relating to use of the addictive substance by the human.

15. The method of claim 12, wherein the one or more doses is selected from the group consisting of a one-time dose, a continuous dose, an intermittent dose and a dose on an as-needed basis.
